(19) United States
(12) Patent Application Publication
(10) Pub. No.: US 2003/0065186 A1
(43) Pub. Date: Apr. 3, 2003

(54) Novel 4-diphenylethyl piperidine derivatives

(76) Inventors: Kah Hiing Ling, Overland Park, KS (US); Paul Wayne Brown, Leawood, KS (US); Robert M. Strom, Midland, MI (US); Richard A. Wolf, Midland, MI (US)

Correspondence Address: ROSS J. OEHLER, AVENTIS PHARMACEUTICALS INC., ROUTE 202-206, MAIL CODE: D-303A, BRIDGEWATER, PA, 08807, US

(21) Appl. No.: 10/263,869

(22) Filed: Oct. 3, 2002

Related U.S. Application Data
(63) Continuation of application No. 09/968,961, filed on Oct. 2, 2001, now abandoned.
(63) Continuation of application No. 09/553,903, filed on Apr. 20, 2000, now abandoned.
(63) Continuation of application No. 09/378,016, filed on Aug. 19, 1999, now abandoned.
(63) Continuation of application No. 09/206,617, filed on Dec. 7, 1998, now abandoned.
(63) Continuation of application No. 08/795,965, filed on Feb. 6, 1997, now abandoned.
Application No. 08/795,965 is a continuation of application No. 08/436,535, filed on May 8, 1995, now abandoned.

(51) Int. Cl. C07D 211/34; C07D 211/22

(52) U.S. Cl. 546/238; 546/241

(57) ABSTRACT

This invention relates to novel substituted piperidine derivatives. More particularly, this invention relates to substituted phenyl 4-substituted-piperidinoalkanol derivatives which are useful as antihistamines, antiallergy agents and bronchodilators.

### BACKGROUND OF THE INVENTION

This invention relates to novel diphenylmethyl piperidine derivatives. More particularly, this invention relates to 4-diphenylmethyl piperidinobutanol derivatives which are useful as antihistamines, antiallergy agents and bronchodilators.

### SUMMARY OF THE INVENTION

More specifically this invention relates to compounds of formula (I)

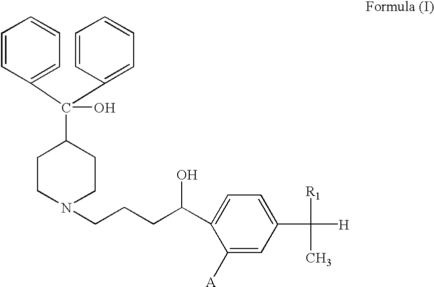

wherein R₁ is —CH₃, —CH₂OH, —COOH or —COO—(C₁₋₆)alkyl; A is hydrogen or hydroxy,

including the stereoisomers, enantiomers, racemic mixtures thereof or their pharmaceutically acceptable salts thereof.

The present invention further provides a method for treating allergic reactions in a patient in need thereof which comprises administering to said patient an effective antiallergic or antihistaminic amount of compound of formula (I).

As used herein in this application:

(a) the term “alkyl” means univalent radical (—R). It includes the straight and branched chain saturated aliphatic hydrocarbyl moieties having the indicated number of carbon atoms. For example, the term “C₁₋₆ alkyl” refers to a saturated straight or branched chain hydrocarbon radical having from one to six carbon atoms, preferably having one to four carbon atoms (“C₁₋₄ alkyl”) and more preferably having one to three carbon atoms (“C₁₋₃ alkyl”). Included within the scope of this term are methyl, ethyl, n-propyl, isopropyl, n-butyl, isobutyl, tertiary butyl, pentyl, isopentyl, hexyl, 2,3-dimethyl-2-butyl, and the like;

(b) the designation —C(O)— or —CO— refers to a carbonyl group of the formula:

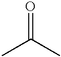

The term —COOR includes those alkoxycarbonyl moieties wherein R is H or a C₁₋₆ alkyl moiety or preferably a C₁₋₃ alkyl moiety, embracing, for example, methoxycarbonyl, ethoxycarbonyl, t-butyloxycarbonyl, and the like. It is also understood that an alkoxycarbonyl wherein R is other than H is also referred to as an ester;

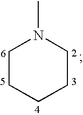

(d) the term “halo” refers to a halogen such as a fluorine atom a chlorine atom or a bromine atom, or a iodine atom.

The term “pharmaceutically acceptable salts” include those acid addition salts derived by reaction with acids, for example, hydrochloric, hydrobromic, sulfuric, nitric or phosphoric acids and such organic carboxylic acids as acetic, propionic, glycolic, maleic, tartaric, citric, salicylic, 2-acetyloxybenzoic acids or organic sulfonic acids such as methanesulfonic, 4-toluenesulfonic and naphthalenesulfonic acids. Of course other acids well known to the pharmaceutical art may also be utilized. The term “pharmaceutically acceptable salts” may also include hydrates.

Stereoisomers of the compounds of formula (I) is a general term for all isomers of these compounds that differ only in the orientation of their atoms in space. It includes geometric (cis/trans) isomers, and isomers of compounds with more than one chiral center that are not mirror images of one another (diastereomers or diastereoisomers). The term “enantiomer” refers to two stereoisomers that are mirror images of one another and not identical, not being superposable. The term “chiral center” refers to a carbon atom to which four different groups are attached. The nomenclature R/S is used as described in IUPAC-IUB Joint Commission on Biochemical Nomenclature, Eur. J. Biochem. 138: 9-37 (1984). A chiral material may either contain an equal amount of the R and S isomers in which case it is called “racemic mixture” or it may not contain equal amounts of R and S isomer in which case it is called “optically active”, or “nonracemic mixture”. A mixture may be resolved or isolated according to conventional and standard procedures well known in the art, e.g., chromatographic separation on chiral stationary phase, use of optically active esters, fractional crystallization of addition salts formed by reagents used for that purpose, as described in “Enantiomers, Racemates, and resolutions”, J. Jacques, A. Collet, and S. H. Wilen, Wiley (1981), enzymatic resolution and the like. Stereoisomer resolution is carried out on the intermediates, or the final products of formula (I). The term “resolution” means separation of a racemic mixture into its optically active components. In addition, enantiomers may be prepared by utilizing enantioselective or asymmetric synthesis which are well known by a person of ordinary skill in the art. The term “enantioselective” or “asymmetric” means the ability to produce a product in an optically active form.

It is understood that the compounds of formula (I) may exist in a variety of stereoisomeric configurations. It is further understood that the compounds of the present invention encompass those compounds of formula (I) in each of their various structural and stereoisomeric configurations as individual isomers or as mixtures of isomers.

The compounds of this invention are prepared by various means, and certain compounds of the invention are employed to prepare other compounds of the invention.

The compounds of the formula (I) may be synthesized by one with ordinary skill in the art using the procedures as more fully described in the following U.S. Pat. No. 4,254,129 issued Mar. 3, 1981 and U.S. Pat. No. 4,254,130 issued Mar. 3, 1981 which are incorporated herein by reference.

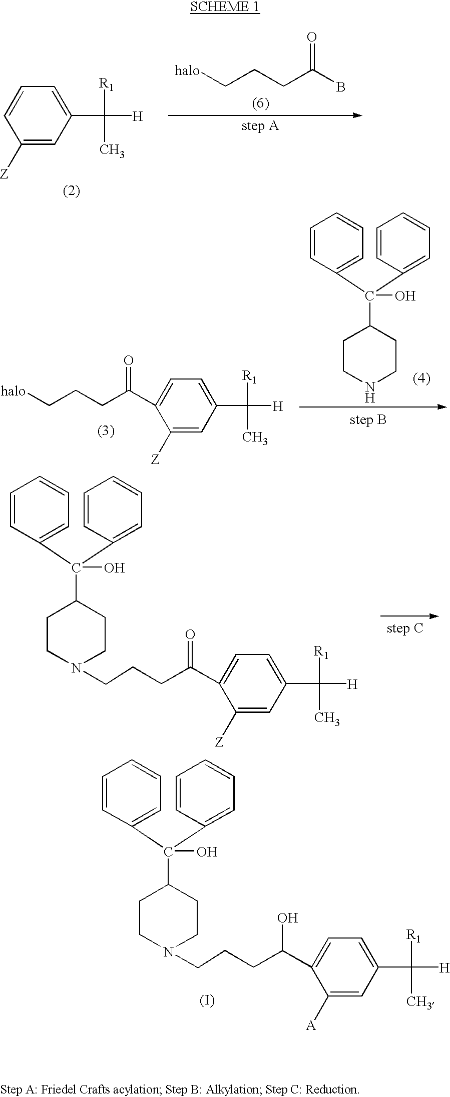

### Scheme 1

Generally, the compounds of formula (I) wherein R₁ is —CH₃, —COOH, or —COO—(C₁₋₆ alkyl) may be synthesized following the general scheme 1.

Step A

The ω-halo phenylbutanone derivative of structure (3), wherein Z is hydrogen, hydroxy or a protected hydroxy, may be prepared by reacting an appropriate phenyl derivative of formula (2), wherein Z is hydrogen, hydroxy or a protected hydroxy, with an appropriate ω-halo compound of the structure (6) halo-(CH₂)₃—C(═O)—B, wherein B is halo or hydroxy, halo is Cl, Br or I, which is known in the art or prepared by procedures well known in the art, under general conditions of a Friedel Crafts acylation as disclosed in Methoden der Organischen Chemie (Houden-Weyl, VII/2a teil I, 1973); or in Friedel-Crafts and related reactions (Interscience, New York, 1963-1964), which are incorporated herein by reference. The reaction is carried out most commonly in a solvent such as methylene chloride, dichloroethane, tetrachloroethane, chlorobenzene, nitromethane, 1-nitropropane, diethyl ether, acetonitrile, n-hexane or carbon disulfide or without any solvent in the presence of a suitable Lewis acid such as ferric chloride, iodine, zinc chloride, aluminum chloride and iron. More preferably the reaction is carried out using methylene chloride as solvent and aluminum chloride or ferric chloride as catalyst. The reaction time varies from ½ hour to 25 hours, preferably 4 to 10 hours and the reaction temperature varies from −15° C. to 100° C., preferably from −10° C. to 20° C. The corresponding ω-halo phenylbutanone derivative of structure (3) is recovered from the reaction zone by an aqueous quench followed by extraction as known in the art. The ω-halo phenylbutanone derivative of structure (3) may be purified by procedures well known in the art, such as crystallization and/or distillation.

Step B

The diphenylmethyl piperidine oxobutyl derivative of formula (5) is obtained by alkylation of 4(α,α-diphenyl) piperidine methanol of formula (4) with an ω-haloalkyl phenylbutanone derivative of formula (3) wherein halo is Cl, Br or I and Z is hydrogen or hydroxy or protected hydroxy as described in U.S. Pat. No. 4,254,130. The alkylation reaction is carried out in a suitable solvent, preferably in the presence of a suitable non-nucleophilic base and optionally in the presence of a catalytic amount of an iodide source, such as potassium or sodium iodide. The reaction time varies from about 4 to 120 hours and the reaction temperature varies from about 40° C. to the reflux temperature of the solvent. Suitable solvents for the alkylation reaction include alcohol solvents such as, methanol, ethanol, isopropyl alcohol, or n-butanol; ketone solvents, such as, cyclohexanone, methyl isobutyl ketone; hydrocarbon solvents, such as, benzene, toluene or xylenes; halogenated hydrocarbons, such as, chlorobenzene or methylene chloride or dimethylformamide. More preferably a mixture of water and hydrocarbon solvents, such as xylenes, is used. Suitable non-nucleophilic bases for the alkylation reaction include inorganic bases, for example, sodium bicarbonate, potassium carbonate, or potassium bicarbonate or organic bases, such as, a trialkylamine, for example, triethylamine or pyridine, or an excess of 4(α,α-diphenyl) piperidine methanol of formula (4) may be used.

The desired compound of formula (I) may be prepared in one step by reduction of the so-produced ketone (5) or in two steps by reduction of the ketone (5) followed by base hydrolysis, or in two steps by base hydrolysis followed by reduction of the ketone (5), depending on the compound desired and the reducing agent employed as disclosed in U.S. Pat. No. 4,285,957.

For example, reduction of the appropriate diphenyl-methyl piperidine oxobutyl derivative of structure (5) wherein R₁ is —CH₃ or —COO—(C₁₋₆ alkyl), using, for example, a suitable reducing agent such as sodium borohydride, potassium borohydride, sodium cyanoborohydride, or tetramethylammonium borohydride is carried out in lower alcohol solvents, such as, methanol, ethanol, isopropyl alcohol or n-butanol, or in aqueous lower alcohol solutions, at temperatures ranging from about 0° C. to the reflux temperature of the solvent, and the reaction time varies from about ½ hour to 8 hours. Preferably, the reaction is carried out using sodium borohydride or potassium borohydride as reducing agent, in presence of sodium hydroxide in an aqueous solution of alcohol such as methanol or ethanol. Other suitable reducing agents are, for example, lithium tri-tert-butylaluminohydride and diisobutylaluminum hydride. These reduction reactions are carried out in suitable solvents diethyl ether, tetrahydrofuran or dioxane at temperatures ranging from about 0° C. to the reflux temperature of the solvent, and the reaction time varies from about ½ hour to 8 hours.

Catalytic reduction may also be employed in the preparation of appropriate diphenylmethyl piperidine derivative of structure (I) wherein R₁ is —CH₃ or —COO—(C₁₋₆ alkyl) from an appropriate diphenylmethyl piperidine oxobutyl derivative of structure (5) wherein R₁ is —CH₃ or COO—(C₁₋₆ alkyl), using hydrogen gas in the presence of a suitable catalyst such as Raney nickel, palladium, platinum or rhodium catalysts in lower alcohol solvents, such as, methanol, ethanol, isopropyl alcohol or n-butanol or acetic acid or their aqueous mixtures, or by the use of aluminum isopropoxide in isopropyl alcohol.

Reduction using sodium borohydride or potassium borohydride is preferred over catalytic reduction for those diphenylmethyl piperidine derivatives of structure (I) wherein R₁ is —CH₃ or —COO—(C₁₋₆ alkyl).

In addition, a chiral reduction of the appropriate diphenylmethyl piperidine oxobutyl derivative of structure (5) wherein R₁ is —CH₃ or —COO—(C₁₋₆ alkyl), using, for example, (+) or (−)-B-chlorodiisopinocamphenylborane gives the corresponding (R) or (S)-diphenylmethyl piperidine derivative of structure (I) wherein R₁ is —CH₃ or —COO—(C₁₋₆ alkyl). Other suitable chiral reducing agents are, (R) and (S)-oxazaborolidine/BH₃, potassium 9-O-(1,2:5,6-di-O-isopropylidine-α-D-glucofuransoyl)-9-boratabicyclo[3.3.1]-nonane, (R) and (S)-B-3-pinanyl-9-borabicyclo[3.3.1]nonane, NB-Enantride, Lithium (R)-(+) and (S)-(−)-2,2′-dihydroxy-1,1′-binaphthyl alkoxyl aluminum hydride, (R)-(+) and (S)-(−)-2,2′-dihydroxy-6,6′-dimethylbiphenyl borane-amine complex, tris[[(1S,2S,5R)-2-isopropyl-5-methyl-cyclohex-1-yl]methyl]aluminum, [[(1R,3R)-2,2-dimethylbicyclo[2.2.1]-hept-3-yl]methyl]beryllium chloride, (R)-BINAP-ruthenium complex/H₂ and 6,6′-bis(diphenylphosphino)-3,3′-dimethoxy-2,2′,4,4′-tetramethyl-1,1′-biphenyl.

The compounds wherein R₁ is —COO—(C₁₋₆ alkyl) may be hydrolyzed by treatment with an inorganic base to give the corresponding diphenylmethyl piperidine derivative of formula (I) R₁ is —COOH.

For example, hydrolysis may be achieved by using a suitable non-nucleophilic base, such as sodium methoxide in methanol as is known in the art. Other methods known in the art for ester cleavage include potassium carbonate in methanol, methanolic ammonia, potassium carbonate, potassium hydroxide, calcium hydroxide, sodium hydroxide, magnesium hydroxide, sodium hydroxide/pyridine in methanol, potassium cyanide in ethanol and sodium hydroxide in aqueous alcohols, with potassium hydroxide being preferred. The reaction is typically carried out in an aqueous lower alcohol solvent, such as methanol, ethanol, isopropyl alcohol, n-butanol, 2-ethoxyethanol or ethylene glycol or pyridine, at temperatures ranging from room temperature to the reflux temperature of the solvent, and the reaction time varies from about ½ hour to 100 hours.

The diphenylmethyl piperidine derivative of formula (I) wherein R₁ is —CH₂OH may be prepared by reducing the corresponding derivative wherein R₁ is —COOH or —COO—(C₁₋₆ alkyl).

For example, reduction of the appropriate diphenyl-methyl piperidine oxobutyl derivative of structure (5) wherein R₁ is —CH₂OH, using, for example, a suitable reducing agent such as lithium aluminum hydride or diborane is carried out in ether solvents such as, for example, diethyl ether, tetrahydrofuran or dioxane at temperatures ranging from about 0° C. to the reflux temperature of the solvent, and the reaction time varies from about ½ hour to 8 hours.

In addition, the individual (R) and (S) isomers of the diphenylmethyl piperidine derivative of formula (I) can be prepared by techniques and procedures well known and appreciated by one of ordinary skill in the art.

For example, the mixture of (R) and (S) isomers of the diphenylmethyl piperidine derivative of formula (I) may be subjected to chiral chromatography to give the corresponding individual (R)-diphenylmethyl piperidine derivative of formula (I) and (S)-diphenylmethyl piperidine derivative of formula (I).

In addition, the individual (R) and (S) isomers of the diphenylmethyl piperidine oxobutyl derivative of formula (5) and the diphenylmethyl piperidine derivative of formula (I) can be prepared by techniques and procedures well known and appreciated by one of ordinary skill in the art and described in “Enantiomers, Racemates, and Resolutions”, Jacques, Collet and Wilen, Wiley (1981).

One such method involves reacting the mixture of (R) and (S) isomers of the diphenylmethyl piperidine derivative of formula (I) with appropriate chiral acids to give the corresponding mixture of diastereomeric acid addition salts. The individual (R)-chiral acid addition salts of the diphenylmethyl piperidine compound of structure (I) and (S)-chiral acid addition salts of the diphenylmethyl piperidine compound of structure (I) are obtained by recrystallization and the individual chiral (R)-diphenylmethyl piperidine compound of structure (I) and chiral (S)-diphenylmethyl piperidine compound of structure (I) are obtained by subjecting the individual (R)-chiral acid addition salts of the diphenylmethyl piperidine compound of structure (I) and (S)-chiral acid addition salts of the diphenylmethyl piperidine compound of structure (I) to base in order to free the piperidine nitrogen from the acid addition complex. Examples of suitable chiral acids are tartaric acid (+), (−), O,O′-dibenzoyltartaric acid (+), (−), O,O′-di-p-toluyltartaric acid (+), (−), 2-Nitrotartranillic acid (+), (−), mandelic acid (+), (−), malic acid (+), (−), 2-phenoxypropionic acid (+), hydratropic acid (+), (−), N-acetylleucine (−), (+), N-(α-methylbenzyl)succinamide (+), (−), N-(α-methylbenzyl)-phthalamic acid (+), (−), camphor-10-sulfonic acid (+), 3-bromocamphor-9-sulfonic acid (+), (−), camphor-3-sulfonic acid (+), quinic acid (+), (−), Di-O-isopropylidene-2-oxo-L-gulonic acid (−), Lasalocid (−), 1,1′-binaphthyl-2,2′-phosphoric acid (+) (−), chloestenonesulfonic acid.

In addition, the individual (R) and (S) isomers of the diphenylmethyl piperidine derivative of formula (I) can be prepared by reacting the mixture of (R) and (S) isomers of the diphenylmethyl piperidine derivative of formula (I) with appropriate organic chiral acids to give the corresponding mixture of diastereomeric acid esters. The individual chiral ester of (R)-diphenylmethyl piperidine compound of structure (I) and chiral ester of (S)-diphenylmethyl piperidine compound of structure (I) are obtained by recrystallization or chromatography and the individual chiral (R)-diphenylmethyl piperidine compound of structure (I) and chiral (S)-diphenylmethyl piperidine compound of structure (I) are obtained by subjecting chiral ester of (R)-diphenylmethyl piperidine compound of structure (I) and chiral ester of (S)-diphenylmethyl piperidine compound of structure (I) to hydrolysis conditions.

It is understood that each hydroxy group in the compounds described in this invention are optionally protected or unprotected. The selection of and utilization of suitable protecting groups is well known by one with ordinary skill in the art and is described in “Protective Groups In Organic Chemistry”, Theodora W. Greene, Wiley (1981) which is herein incorporated by reference. For example, suitable protecting group for those hydroxy functionalities present include ethers such as methyl ether, cyclohexyl ether, isopropyl ether, t-butyl ether, or methoxymethyl ether, tetrahydropyranyl, tetrahydrothio-furanyl, 2-phenylselenylethyl ether, o-nitrobenzyl ether, trimethylsilyl ether, t-butyldiphenylsilyl ether, tribenzylsilyl ether, isopropyldimethylsilyl ether, t-butyldimethyl silyl ether, t-butyldiphenylsilyl ether, tribenzylsilyl ether, triisopropylsilyl ether; and ester, such as acetate ester, levulinate ester (CH₃COCH₂CH₂CO₂—), pivaloate ester ((CH₃)₃CCO₂—), benzoate ester, 2,4,6,-trimethylbenzoate (mesitoate) ester, methyl carbonate, p-nitrophenyl carbonate, p-nitrobenzyl carbonate, S-benzyl thiocarbonate and N-phenylcarbamate, phosphinates such as dimethyl-phosphonyl ester ((CH₃)₂P(O)O—), sulfonates such as methyl-sulfonate or mesyl (—OSO₂CH₃) or toluene sulfonate or tosyl (—OSO₂C₆H₄—ρ—CH₃).

The 4(α,α-diphenyl) piperidine methanol of structure (4) is readily available to one with ordinary skill in the art and is described in U.S. Pat. No. 4,254,129, Mar. 3, 1981, U.S. Pat. No. 4,254,130, Mar. 3, 1981, U.S. Pat. No. 4,285,958, Apr. 25, 1981 and U.S. Pat. No. 4,550,116, Oct. 29, 1985.

The derivatives of formula (2) are commercially available or readily prepared by one with ordinary skill in the art.

Alternatively, one with ordinary skill in the art may synthesize the compounds of formula (I) by using the procedures disclosed in the PCT application WO93/21156 published Oct. 28, 1993 or in the PCT application WO95/00480 published Jan. 5, 1995 which are herein incorporated by reference.

The following examples present typical syntheses as described in Scheme 1. These examples are understood to be illustrative only and are not intended to limit the scope of the present invention in any way. As used herein, the following terms have the indicated meanings: “g” refers to grams; “mmol” refers to millimoles; “mL” refers to milliliters; “bp” refers to boiling point; “mp” refers to melting point; “° C.” refers to degrees Celsius; “Pa” refers to pascals; “μL” refers to microliters; “μg” refers to micrograms; and “μM” refers to micromolar; “TLC” refers to thin layer chromatography; “M” refers to molarity; “N” refers to normal, “[α]_{D}²⁰” refers to specific rotation of the D line of sodium at 20° C. obtained in a 1 decimeter cell; “GC” refers to gas chromatography; “R_{f}” refers to retention factor and “RPM” refers to revolutions per minute.

### EXAMPLE 1

### Ethyl 4-[4-[4-(Hydroxydiphenylmethyl)-1-Piperidinyl]-1-Hydroxybutyl]α-Methylphenyl Acetate

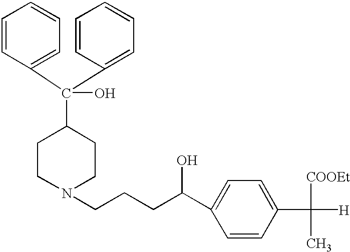

Step 1

Ethyl 2-Phenylpropionic Acid Ester

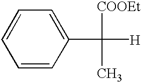

Load into a round-bottomed flask equipped with a condenser and a magnesium sulfate drying tube on top, 2-phenyl propionic acid (1.51 mol, 226 g), concentrated sulfuric acid (3.32 g, 0.033 mol) and absolute ethanol (1 L). Heat the resulting solution at reflux for 22.5 hours. Concentrate the solution under vacuum to obtain an oil (277 g). Add to the oil one liter of fresh ethanol and heat the resulting solution at reflux for another 19.6 hours. Add to the reaction, at ambient temperature; sodium ethoxide (21 weight percent in ethanol, 30 mL). Then add glacial acetic acid (2 g) in order to establish a slightly acidic pH. Remove the solids from the slurry by suction filtration. Concentrate the filtrate under vacuum on a rotary evaporator. Add heptane (400 mL) to the residue and concentrate this solution under vacuum in order to strip away remaining traces of ethanol to give ethyl 2-phenylpropionic acid ester as an oil (276.7 g).

Step 2

Ethyl 4-(4-Chloro-1-Oxobutyl)-α-Methylphenyl Acetate

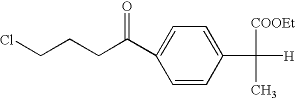

Load into a round-bottomed flask equipped with a condenser having a magnesium sulfate drying tube at the top, aluminum chloride (458 g, 3.44 mol) and methylene chloride (200 mL). Stir the resulting slurry at 250 RPM and cool to 2° C. via ice/water bath. Add to the cold slurry 4-chlorobutyryl chloride (210 mL, 1.87 mol), and methylene chloride (20 mL) over a 40 minute period so as to keep the temperature of the slurry below 15° C. Cool the slurry again to 2° C. and add ethyl 2-phenylpropionic acid ester (276.7 g, 1.55 mol) by addition funnel over a period of 70 minutes so as to keep the temperature of the solution below 15° C. Add methylene chloride (100 mL) as to rinse. Allow the solution to warm to ambient temperature over a 80 minute period. Heat the solution from 22 to 42° C. over a 3.3 hour period.

Put ice (1.5 kg) into a 4 L beaker. Pour into this ice with stirring about one-half of the methylene reaction (500 mL). Stir for 10 minutes and add a second volume of methylene chloride (100 mL). Filter the organic and aqueous solution by suction through a pad of filteraid on a coarse sintered glass funnel. Separate the organic and aqueous phases and extract the aqueous phase with methylene chloride (200 mL). Add the methylene chloride to the organic layer. Work up the other half of the unquenched methylene chloride solution in a similar fashion.

Concentrate the combined organic layers under vacuum, up to 90° C. at 25 mm Hg (3.33 kPa), to give a brown oil and solids (465.4 g). Add ethanol (300 mL) to the mixture. Put the resulting solution into a round-bottomed flask, fitted with an overhead stirrer, a reflux condenser (with a drying tube on the top) and a gas sparge tube. Sparge anhydrous hydrogen chloride (22.25 g, 0.61 mol) into the stirred solution. Heat the solution, to 56° C., over a 3.75 hour period with stirring. Add to the solution at 56° C. sodium ethoxide (21 weight percent in absolute ethanol; 835 g, 2.58 mol sodium ethoxide) over a period of 100 minutes. Heat the resulting liquid/solid slurry over a period of 15 minutes at 52° C. Cool the solution to below 20° C. by ice/water bath. Add to the slurry glacial acetic acid (25.5 mL, 0.445 mol) (pH of an aliquot diluted with an equal volume of water is 5.0-5.2). Add heptane (250 mL) and allow the slurry to stand at ambient temperature overnight.

Filter by suction through a pad of filteraid on a coarse sintered glass funnel. Wash the filtercake with heptane/absolute ethanol (400 mL, 2/1 (v/v)). Concentrate the combined filtrate and washes on a rotary evaporator up to 95° C. at 110 mm Hg (14.3 kPa), to obtain brown liquid and solid residues (433 g). Flash distill the residue through a bump guard and Claisen head with no rectification at 1 mm Hg vacuum. Collect distillate at overhead temperatures of 40-175° C. to obtain a light yellow oil (346.7 g). Discard the distillation pot. Purify the so-produced oil as a mixture of ethyl 3- and 4-(cyclopropylcarbonyl)-α-methylphenyl acetate by flash distillation under vacuum through a 1 inch I.D. column, length of 53 inches, packed with 316 stainless steel High Goodloe 773. Collect the desired para derivative ethyl 4-(cyclopropylcarbonyl)-α-methylphenyl acetate (95.9 g) at overhead of 146-147° C. temperatures.

Put ethyl 4-(cyclopropylcarbonyl)-α-methylphenyl acetate (73.89 g, 0.300 mol), mixed xylenes (400 mL) and absolute ethanol (90 mL) into a round-bottomed flask fitted with an overhead paddle stirrer, a gas sparge tube with fritted end and a reflux condenser with a magnesium sulfate drying tube. Sparge hydrogen chloride gas from a lecture bottle (36.68 g, 1.061 mol, anhydrous 99%) into the stirred solution over a period of 15 minutes. Replace the gas sparge tube with a glass stopper. Heat the solution with stirring, the temperature rising from 40° C. to 79° C. in 45 minutes. Maintain the temperature at 79° C. for another 15 minutes. Replace the reflux condenser with a simple still head fitted with a condenser and a thermometer . Distill and collect at overhead temperature (80-138° C.). Allow the yellow solution to cool to ambient temperature and remove the xylene solvents by rotary evaporation up to 75° C. at 12 mm Hg (1.6 kPa) to leave the ethyl 4-(4-chloro-1-oxobutyl)-α-methylphenyl acetate (87.4 g) as a yellow solid.

Step 3

Ethyl 4-[4-[4-(Hydroxydiphenylmethyl)-1-Piperidinyl]-1-Oxobutyl]-α-Methylphenyl Acetate

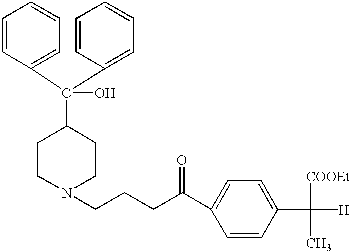

Add ethyl 4-(4-chloro-1-oxobutyl)-α-methylphenyl acetate (7.6 g, 26.9 mmol) to a solution of 4(α,α-diphenyl)piperidine methanol (15.8 g, 59.0 mmol) in xylenes (27 mL) into a single neck round-bottomed flask equipped with a water-cooled reflux condenser and on the outlet a calcium sulfate-filled drying tube. Stir and heat the reaction at 140° C. for 5.5 hours. Cool the slurry reaction to ambient temperature and add xylenes (15 mL). Heat the diluted slurry reaction at 50° C. and add glacial acetic acid (1.52 g, 25.3 mmol). Cool the reaction to ambient temperature and filter by suction. Wash the filtercake with xylenes (25 mL) and add the filtrate wash to the original filtrate.

Stir filtrate at ambient temperature and add 37% aqueous hydrochloric acid (3.02 g, 30.6 mmol) over a 70 min period, to provide a thick solid/liquid slurry. Add to the slurry absolute 2B ethanol (3 mL) and stir the resulting slurry for 10 min. Collect the solids by suction filtration, and wash the filtercake with fresh xylenes (20 mL) and heptane (10 mL). Dry the filtercake overnight in a vacuum oven at 47° C. to obtain 11.05 g of crude ethyl 4-[4-[4-(hydroxydiphenylmethyl)-1-piperidinyl]-1-oxobutyl]-α-methylphenyl acetate as a light tan solid.

Reduce the so-produced 4-(4-chloro-1-oxobutyl)-α-methylphenyl acetate following the procedure described in Example 4, step 3 to give the corresponding ethyl 4-(4-chloro-1-hydroxybutyl)-α-methylphenyl acetate.

### EXAMPLE 2

### 4-[4-[4-(Hydroxydiphenylmethyl)-1-Piperidinyl]-1-Hydroxybutyl]-α-Methylphenyl Acetic Acid

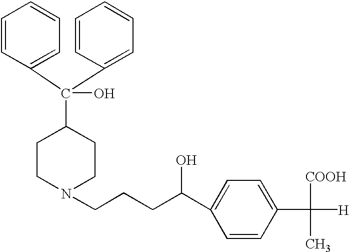

Add ethyl 4-[4-[4-(hydroxydiphenylmethyl)-1-piperidinyl]-1-oxobutyl]-α-methylphenyl acetate (6.00 g, 10.5 mmol) to a solution of methanol (30 mL), 50% aqueous sodium hydroxide (4.30 g, 53.8 mmol) and water (3.5 g) Heat under reflux for 1.75 hours. Dissolve the forming solids by addition of water (6 mL). Cool the reaction to 41° C. and add sodium borohydride (0.22 g, 5.82 mmol). Stir the reaction at 40° C. for 1.83 hours. Add acetone (1.65 mL, 22.5 mmol) to the solution and stir at 40° C. for 0.5 hour and overnight at ambient temperature. Heat the solution to 32° C. and add 37% aqueous hydrochloric acid (6.66 g, 67.6 mmol) and 5% aqueous hydrochloric acid (7.10 g, 9.7 mmol) in order to reduce the pH of the solution to 2.0.

Add water (24 g) and heat the resulting solution to 37° C. Cool the solution slowly to −20° C. and collect solids by suction filtration. Wash the filtercake with cold water (10 mL) and dry it at 52° C. for 70 min under vacuum to obtain 4-[4-[4-(hydroxydiphenylmethyl)-1-piperidinyl]-1-oxobutyl]-α-methylphenyl acetic acid hydrate as a white solid (5.85 g). Add the so-produced hydrate (5.00 g) to a solution of acetone (15 mL) and water (0.56 g). Stir the mixture at ambient temperature until almost all the solids are dissolved. Filter the solution through a filter aid by suction to obtain a clear solution and rinse with acetone (2 mL). Transfer the filtrate in a single-neck, round-bottomed flask using acetone (13 mL). Stir and heat under reflux. Add ethyl acetate (30 mL) slowly to the refluxing solution, a second liquid phase appears after 12 mL of ethyl acetate has been added. Stir the liquid/liquid mixture at ambient temperature overnight. Reheat the mixture for one hour at reflux and cool to 40° C. Remove the supernatant solvent phase by pipette. Add fresh acetone (30 mL) and heat the solution under reflux. Add ethyl acetate (30 mL) to the refluxing solution over a 45 min period. Break up the solids by spatula. Heat the resulting slurry under reflux for another hour and then cool to ambient temperature. Collect the solid by suction filtration and wash the the filtercake with ethyl acetate (10 mL). Dry the filtercake in a vacuum oven at 55° C. and dry open to air overnight to obtain anhydrous 4-[4-[4-(hydroxydiphenylmethyl)-1-piperidinyl]-1-hydroxybutyl]-α- methylphenyl acetic acid as a white solid (3.09 g, 63%).

### EXAMPLE 7

### 4-[4-[4-(Hydroxydiphenylmethyl)-1-Piperidinyl]-1-Hydroxybutyl]-2-Methylphenethyl Alcohol

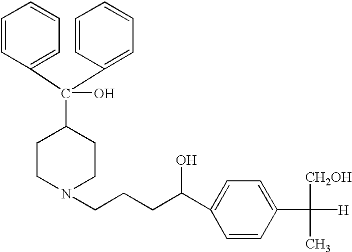

Add a suspension of ethyl 4-[4-[4-(hydroxydiphenyl-methyl)-1-piperidinyl]-1-hydroxybutyl]-α-methylphenyl acetate (4 mmol) in tetrahydrofuran (50 mL) slowly to a suspension of lithium aluminium hydride (18 mmol) in tetrahydrofuran (60 mL) under nitrogen atmosphere with stirring. Stir the mixture and heat under reflux for about 3 hours and add tetrahydrofuran (30 mL). Heat under reflux for 4 hours and let stand overnight (about 16 hours). Stir the mixture under a nitrogen atmosphere and add water (2 mL) cautiously followed by an aqueous solution of sodium hydroxide (10%, 2 mL), water (2 mL) and sodium sulfate (4 g). Warm the mixture to 50-55° C. and stir for 45 minutes, filter and wash the solids and the material with tetrahydrofuran. Combine the filtrates and evaporate under vacuum. Recrystallized the residue from ethanol to give 4-[4-[4-(hydroxydiphenylmethyl)-1-piperidinyl]-1-hydroxybutyl]-2-methylphenethyl alcohol.

### EXAMPLES 4, 5 AND 6

### Ethyl 4-[4-[4-(Hydroxydiphenylmethyl)-1-Piperidinyl]-1-Hydroxybutyl]-α-Methyl-3-Hydroxyphenyl Acetate, 4-[4-[4-(Hydroxydiphenylmethyl)-1-Piperidinyl]-1-Hydroxybutyl]-α-Methyl-3-Hydroxyphenyl Acetic Acid and 4-[4-[4-(Hydroxydiphenylmethyl)-1-Piperidinyl]-1-Hydroxybutyl]-2-Methyl-2-(3-Hydroxyphenyl)-Ethyl Alcohol

can be prepared by one ordinary skilled in the art following the above described examples 1, 2 and 3 but using 2-(3-hydroxyphenyl) propionic acid as starting material instead of 2-phenyl propionic acid. The hydroxy group may be protected, more preferably methoxymethyl ether group is used.

### 2-(3-Hydroxyphenyl) Propionic Acid

Ethyl 2-(3-methoxyphenyl) propionic acetate can be prepared by one with ordinary skill in the art following the procedure described by Sedgeworth et al. in J. Chem. Soc. Perk T1 (12), 2677-2687 (1985) which is herein incorporated by reference. Ethyl 2-(3-methoxyphenyl) propionic acetic ester is further deprotected and hydrolyzed according well known procedures in the art disclosed in “Protective Groups In organic chemistry” which is herein incorporated by reference.

### EXAMPLE 7

### 4-[4-[4- (Hydroxydiphenylmethyl)-1-Piperidinyl]-1-( 4-Isoprpylphenyl) Butanol

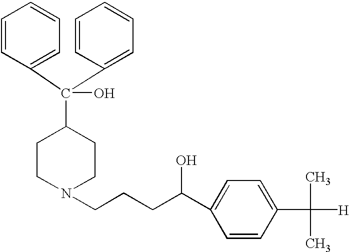

Step 1

1-Chloro-4-(4-Isopropylphenyl) Butanone

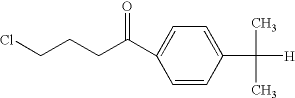

Stir aluminium chloride (501.52 g, 3.76 mol) and methylene chloride (1.4 L) in a round-bottomed flask equipped with a nitrogen bubbler. Cool the resulting slurry to −10° C. via ice/ethanol bath. Add 4-chlorobutyryl chloride (546.05 g, 3.87 mol) over a period of 45 min so as to keep the temperature of the slurry solution below −3° C. Cool the resulting solution to −10° C., and add cumene (477 mL, 3.43 mol) over a period of 80 min, maintaining the temperature of the solution at around −10° C.

Into a 4L beaker with ice (1 kg) and stirring, pour about one-half of the methylene solution above. Stir the mixture for 30 min. Separate the organic and aqueous phases. Wash the organic phase with water (500 mL) and then with an aqueous solution of sodium bicarbonate 1% (500 mL). Work up the other half of the unquenched methylene chloride solution in a similar fashion. Combine the organic phases and concentrate under vacuum. After collection of 1.3 L of methylene chloride solution, add heptane (400 mL) to the residue in order to complete the drying of the isopropyl ketone. Remove the heptane under vacuum to give a yellow oil. Add methanol (700 mL) to this oil and store the solution at −20° C. for 16 hours. Separate the formed solids from the supernatant by decantation. Add hexane (100 mL) and crush the solids in the hexane slurry. Collect the slurry by suction filtration and wash the filter cake with hexane (300 mL). Dry the filtercake solid at under vacuum (1 mm Hg, 0.13 kPa) at ambient temperature to give 1-chloro-4-(4-isopropylphenyl) butanone (561.43 g, 73%).

Step 2

4-[4-[4-(Hydroxydiphenylmethyl)-1-Piperidinyl]-1-(4-Isopropylphenyl) Butanone

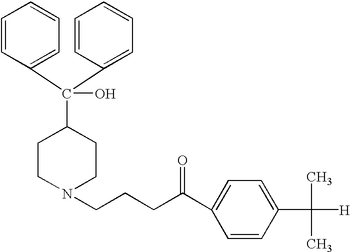

Stir 4(α,α-diphenyl) piperidine methanol hydrochloride (131.0 g, 0.43 mol), potassium carbonate (71.3 g, 0.52 mol) and water (200.0 g) in a round-bottomed flask equipped with a nitrogen bubbler. Add a solution of 1-chloro-4-(4-isopropylphenyl) butanone (129.3 g, 0.58 mol) in warm xylenes (70 mL) to the mixture. Add xylenes (70 mL) to rinse. Heat the mixture to 80° C. for 30 min at 300 RPM then to 100° C. at 300 RPM for one hour then heat 18 hours at 200 RPM.

Add xylenes (150 mL) and stir the resulting mixture for 2 hours at 92° C. Allow the mixture to settle and remove the bottom aqueous phase. Wash the organic phase three times with 140 mL each of water, each time heating above 90° C. during the stirring, settling and decanting operations. Remove some of the xylene solvents by distillation at atmospheric pressure, leaving about 180 mL xylenes remaining in the distillation pot. Cool the solution to 40° C., and add heptane (400 mL). Store the solution at −20° C. for 18 hours to provide a liquid/solid slurry. Collect the solids by suction filtration and wash with heptane (400 mL). Dry the solids under vacuum (1 mm Hg, 0.13 kPa) at ambient temperature to give 4-[4-[4-(hydroxydiphenylmethyl)-1-piperidinyl]-1-(4-isoprpylphenyl) butanone as a white powder (179.16 g, 0.39 mol, 91%).

Step 3

4-[4-[4-(Hydroxydiphenylmethyl)-1-Piperidinyl]-1-(4-Isoprpylphenyl) Butanol

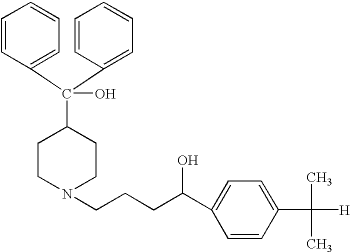

Add 4-[4-[4-(Hydroxydiphenylmethyl)-1-piperidinyl]-1-(4-isoprpylphenyl) butanone (22.78 g, 50 mmol) to a solution of ethanol/water (126 mL, 90/10). Stir and heat the solution under reflux. Add an aqueous solution of sodium borohydride (12%, 24.4 mmol) and sodium hydroxide (40%). Rinse with additional water (10 mL). Heat under reflux for an additional 25 min after the addition is completed. Add water (84 g) to the refluxing solution. Allow the mixture to cool slowly to ambient temperature. Collect the white solid by suction filtration and wash the filtercake with water at ambient temperature (60 mL) and water at 92° C. (115 mL). Dry the solids open to air for three days to obtain 21.76 g. Put the resulting compound (21.00 g) into an erlenmeyer flask with a solution of ethanol and water (150 mL, 90/10). Heat the solution to reflux and then hot polish filter through fluted filter paper. Wash the filter paper with hot ethanol water (25 mL, 90/10). Combine the filtrate and transfer to a 500 mL single-neck, round bottomed flask. Heat under reflux. Add water (36 mL) to obtain some solids. Add absolute ethanol (30 mL) to the refluxing mixture to obtain dissolution of most of all the solids. Allow the mixture to cool to ambient temperature and then to ice/water bath temperature. Collect the resulting white solid by suction filtration, wash the filtercake with ethanol/ water (20 mL, 50/50) and then with cold ethanol/water (24 mL, 50/50). Dry the solids overnight open to air to give 17.50 g (77%) of 4-[4-[4-(hydroxydiphenylmethyl)-1-piperidinyl]-1-(4-isoprpylphenyl) butanol.

### EXAMPLE 8

### 4-[4-[4-(Hydroxydiphenylmethyl)-1-Piperidinyl]-1-( 4-Isoprpyl-3-Hydroxyphenyl) Butanol

may be prepared by one ordinary skilled in the art following the above described example 7 but using 3-isopropyl phenol as starting material instead of cumene. The hydroxy group may be protected, more preferably -o-methoxy methyl group is used.

3-Isopropyl phenol is commercially available.

The compounds of the present invention are useful as antihistamines, antiallergy agents and bronchodilators as more fully described in U.S. Pat. Nos. 4,254,129 issued Mar. 3, 1981 and 4,254,130 issued Mar. 3, 1981.

The compounds can be administered alone or in the form of a pharmaceutical composition in combination with pharmaceutically acceptable carriers or excipients, the proportion and nature of which are determined by the solubility and chemical properties of the compound selected, the chosen route of administration, and standard pharmaceutical practice. The compounds of the invention, while effective themselves, may be formulated and administered in the form of their pharmaceutically acceptable acid addition salts for purposes of stability, convenience of crystallization, increased solubility and the like.

The compounds of this invention can be administered orally, parenterally, for example, subcutaneously, intravenously, intramuscularly, intraperitoneally, by intranasal instillation or by application to mucous membranes, such as, that of the nose, throat and bronchial tubes, for example, in an aerosol spray containing small particles of a compound of this invention in a spray or dry powder form. One skilled in the art of preparing formulations can readily select the proper form and mode of administration depending upon the particular characteristics of the compound selected, the disorder to be treated, the stage of the disorder, and other relevant circumstances.

The compounds of the present invention may be enclosed in gelatin capsules or compressed into tablets. For the purpose of oral therapeutic administration, the compounds may be incorporated with excipients and used in the form of tablets, troches, capsules, elixirs, suspensions, syrups, wafers, chewing gums and the like. These preparations should contain at least 4% of the compound of the invention, the active ingredient, but may be varied depending upon the particular form and may conveniently be between 4% to about 70% of the weight of the unit. The amount of the compound present in compositions is such that a suitable dosage will be obtained. Preferred compositions and preparations according to the present invention are prepared so that an oral dosage unit form contains between 5.0-300 milligrams of a compound of the invention.

The tablets, pills, capsules, troches and the like may also contain one or more of the following adjuvants: binders such as microcrystalline cellulose, gum tragacanth or gelatin; excipients such as starch or lactose, disintegrating agents such as alginic acid, Primogel, corn starch and the like; lubricants such as magnesium stearate or Sterotex; glidants such as colloidal silicon dioxide; and sweetening agents such as sucrose or saccharin may be added or a flavoring agent such as peppermint, methyl salicylate or orange flavoring. When the dosage unit form is a capsule, it may contain, in addition to materials of the above type, a liquid carrier such as polyethylene glycol or a fatty oil. Other dosage unit forms may contain other various materials which modify the physical form of the dosage unit, for example, as coatings. Thus, tablets or pills may be coated with sugar, shellac, or other enteric coating agents. A syrup may contain, in addition to the present compounds, sucrose as a sweetening agent and certain preservatives, dyes and colorings and flavors. Materials used in preparing these various compositions should be pharmaceutically pure and non-toxic in the amounts used.

For the purpose of parenteral therapeutic administration, including topical administration, the compounds of the present invention may be incorporated into a solution or suspension. These preparations should contain at least 0.1% of a compound of the invention, but may be varied to be between 0.1 and about 50% of the weight thereof. The amount of the inventive compound present in such compositions is such that a suitable dosage will be obtained. Preferred compositions and preparations according to the present invention are prepared so that a parenteral dosage unit contains between 5.0 to 100 milligrams of the compound of the invention.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials made of glass or plastic.

The quantity of novel compound of formula (I) administered will vary depending on the patient and the mode of administration and can be any effective amount. The quantity of novel compound may vary over a wide range to provide in a unit dosage an effective amount of from about 0.01 to 60 mg/kg of body weight of the patient per day to achieve the desired effect. For example, the desired antihistamine, antiallergy and bronchodilator effects can be obtained by consumption of a unit dosage form such as a tablet containing 1 to 200 mg of a novel compound of this invention taken 1 to 4 times daily.

For use as aerosols the compounds of this invention in solution or suspension may be packaged in a pressurized aerosol container together with suitable propellants, for example hydrocarbon propellants such as propane, butane or isobutane with usual adjuvants as may be necessary or desirable. The compounds also may be administered in a non-pressurized form such as in a nebulizer or atomizer.

The term patient as used herein is taken to mean warm blooded animals, birds, and mammals, for example, humans, cats, dogs, horses, sheep, bovine cows, pigs, lambs, rats, mice and guinea pigs.

In another embodiment, the present invention provides compositions comprising a compound of formula (I) in admixture or otherwise in association with one or more inert carriers. These compositions are useful, for example, as assay standards, as convenient means of making bulk shipments, or as pharmaceutical compositions. An assayable amount of a compound of formula (I) is an amount which is readily measurable by standard assay procedures and techniques as are well known and appreciated by those skilled in the art.

Assayable amounts of a compound of formula (I) will generally vary from about 0.001% to about 75% of the composition by weight. Inert carriers can be any material which does not degrade or otherwise covalently react with a compound of formula (I). Examples of suitable inert carriers are water; aqueous buffers, such as those which are generally useful in High Performance Liquid Chromatography (HPLC) analysis; organic solvents, such as acetonitrile, ethyl acetate, hexane and the like; and pharmaceutically acceptable carriers or excipients.

More particularly, the present invention provides pharmaceutical compositions comprising an effective amount of a compound of formula (I) in admixture or otherwise in association with one or more pharmaceutically acceptable carriers or excipients.

An effective amount of a compound of formula (I) refers to an amount which is effective, upon single or multiple dose administration to the patient, in providing the desired antihistaminic, antiallergic or bronchodilator effects beyond that expected in the absence of such treatment.

An effective amount of a compound of formula (I), such as an effective antiallergic amount, or an effective antihistaminic amount, can be readily determined by the attending diagnostician, as one skilled in the art, by the use of known techniques and by observing results obtained under analogous circumstances. In determining the effective amount or dose, a number of factors are considered by the attending diagnostician, including, but not limited to: the species of mammal; its size, age, and general health; the response of the individual patient; the particular compound administered; the mode of administration; the bioavailability characteristics of the preparation administered; the dose regimen selected; the use of concomitant medication; and other relevant circumstances.

Treating a patient means to prevent or to alleviate the patient's disease or condition.

As it is true for most classes of compounds suitable or use as therapeutic agents certain subclasses and certain specific compounds are more preferred than others. In this instance it is preferred than A is H, and more preferably A is H and R₁ is —CH₃ or —COOH.

What is claimed is:

1. A compound of the formula 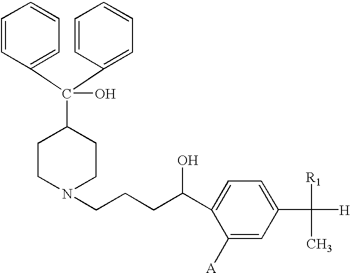
wherein R₁ is —CH₃, —CH₂OH, —COOH or —COO—(C₁₋₆)alkyl; and A is hydrogen or hydroxy;
including the stereoisomers, enantiomers, racemic mixtures thereof or their pharmaceutically acceptable salts thereof.

2. A compound according to claim 1 wherein A is H.

3. A compound according to claim 1 wherein A is OH.

4. A compound according to claim 1 wherein R₁ is —CH_{3.}

5. A compound according to claim 1 wherein R₁ is —CH₂OH.

6. A compound according to claim 1 wherein R₁ is —COOH.

7. A compound according to claim 1 wherein R₁ is —COO—(C₁₋₆)alkyl.

8. A compound according to claim 1 wherein R₁ is —COO—(C₁₋₃)alkyl.

9. A compound according to claim 1 wherein the compound is 4-[4-[4-(hydroxydiphenylmethyl)-1-piperidinyl]-1-hydroxybutyl]-α-methylphenyl acetic acid.

10. A compound according to claim 1 wherein the compound is ethyl 4-[4-[4-(hydroxydiphenylmethyl)-1-piperidinyl]-1-hydroxybutyl]-α-methylphenyl acetate.

11. A compound according to claim 1 wherein the compound is 4-[4-[4-(hydroxydiphenylmethyl)-1-piperidinyl]-1-hydroxybutyl]-2-methylphenethyl alcohol.

12. A compound according to claim 1 wherein the compound is 4-[4-[4-(hydroxydiphenylmethyl)-1-piperidinyl]-1-(4-isoprpylphenyl) butanol.

13. A composition comprising an assayable amount of a compound of claim 1 in admixture or otherwise in association with one or more inert carriers.

14. A pharmaceutical composition comprising an effective antiallergic amount of compound of claim 1 in admixture or otherwise in association with one or more pharmaceutically acceptable carriers or excipients.

15. A pharmaceutical composition comprising an effective antihistaminic amount of compound of claim 1 in admixture or otherwise in association with one or more pharmaceutically acceptable carriers or excipients.

16. A method for treating allergic reactions in a patient in need thereof which comprises administering to said patient an effective antiallergic compound of claim 1.

17. A method for treating allergic reactions in a patient in need thereof which comprises administering to said patient an effective antihistaminic compound of claim 1.
